(19) United States
(12) Plant Patent Application Publication
(10) Pub. No.: US 2023/0060124 P1
(43) Pub. Date: Feb. 23, 2023

(54) HEMP PLANT NAMED 'CAF#13/2020'

(50) ;

(71) Applicant: Creston Aquaporic Spring Farm Inc., Richmond (CA)

(72) Inventors: Ping Zhang, Richmond (CA); Miguel Gallach Caballero, Vienna (AT); Javier Gallach Caballero, Vienna (AT)

(73) Assignee: Creston Aquaponic Spring Farm Inc., Richmond (CA)

(21) Appl. No.: 17/300,106

(22) Filed: Mar. 9, 2021

(51) Int. Cl. A01H 6/28

(52) U.S. Cl. PLT/258

(57) ABSTRACT

A vegetatively reproduced hemp (Cannabis sativa spp.) cultivar named ‘CAF#13/2020’ is distinguished from other commercially produced hemp varieties by its high cannabidiol (CBD) content, low Δ⁹-tetrahydrocannabinol (THC) content, and adaptation to growing conditions in Canada and the northern United States. It is also identifiable by its unique genotype, defined by 845,500 single nucleotide polymorphisms detected against the representative reference genome cs10 (GenBank: GCA_900626175.2; RefSeq: GCF_900626175.2) at the National Center for Biotechnology Information (NCBI).

Latin name: Cannabis sativa.

Variety denomination: ‘CAF#13/2020’.

### BACKGROUND OF THE VARIETY

Cannabis sativa spp. is an annual herbaceous dioecious species, indigenous to the Himalayan Mountains, Central Asia and the Indian subcontinent, and now found worldwide as a result of widespread cultivation. Cannabis sativa spp. has been historically used for a variety of purposes, such as industrial fiber, seed, oil, recreation, food, religious mood and medicine.

The Cannabis sativa spp. is legally divided into two types, which are defined by the content of Δ⁹-tetrahydrocannabinol (THC) in the flowers. The species is called “hemp” when the content of THC is below 0.3% in dry weight; it is called “marijuana” when the THC content is over 0.3% in dry weight.

The growth of the market for cannabidiol (CBD) derived from Cannabis sativa spp. has been significant in recent years, and it is expected to increase. Consequently, there is a high demand in the marketplace for Cannabis sativa spp. varieties with high CBD content and low THC content.

The new hemp plant ‘CAF#13/2020’ was developed in the course of a breeding program carried out by the inventors at Richmond, British Columbia, Canada. The inventors developed a base population of Cannabis sativa plants comprising a pool of hundreds of plants grown from seeds obtained after two generations of random mating, beginning in 2019, between about two dozen hemp varieties owned by the company. The base population then underwent two rounds of selection (with selection intensity 10%), and dozens of randomly chosen plants were selected for a final round of phenotyping. ‘CAF#13/2020’ showed the highest CBD yield (7.380%) in dry flower while having a THC concentration of less than 0.3% (0.277%) by weight. ‘CAF#13/2020’ was first asexually reproduced in 2019 at Richmond, British Columbia, Canada via micro-propagation, and has been observed to remain true to type over successive asexually propagated generations.

### BRIEF DESCRIPTION OF THE VARIETY

‘CAF#13/2020’ is a vegetatively reproduced hemp (Cannabis sativa spp.) cultivar distinguished from other commercially produced hemp varieties by its high CBD content, low THC content, and adaptation to growing conditions in Canada and the northern United States. It is also identifiable by its unique genotype, defined by 845,500 single nucleotide polymorphisms detected against the representative reference genome cs10 (GenBank: GCA_900626175.2; RefSeq: GCF_900626175.2) at the National Center for Biotechnology Information (NCBI).

‘CAF#13/2020’ is distinguished from other known varieties by its relatively high CBD content and low THC content. The claimed variety, the parentage of which is unknown, compares favorably to other low-THC hemp varieties evaluated by the inventors. Table 1 below shows a comparison of the claimed variety to 20 other hemp varieties, selected from among the top 10% of the better strains produced in the inventor's breeding program. The comparison varieties represent those with the most favorable analytical values and morphology as determined by the breeding program goals.

TABLE 1
Comparison of ‘CAF #13/2020’
to Other Known Hemp Varieties

| | CBD | THC |
|---|---|---|
| Sample # | (CBD + CBDA) | (THC + THCA) |
| 1 | 2.97 | 0.191 |
| 2 | 4.66 | 0.206 |
| 3 | 5.90 | 0.232 |
| 4 | 9.54 | 0.326 |
| 5 | 8.27 | 0.355 |
| 6 | 6.62 | 0.297 |
| 7 | 4.09 | 0.195 |
| 8 | 4.15 | 0.219 |
| 9 | 5.90 | 0.227 |
| 10 | 8.69 | 0.364 |
| 11 | 4.15 | 0.178 |
| 12 | 9.34 | 0.355 |
| CAF #13/2020 | 7.38 | 0.277 |
| 14 | 3.17 | 0.164 |
| 15 | 7.56 | 0.312 |
| 16 | 7.94 | 0.367 |
| 17 | 6.60 | 0.257 |
| 18 | 4.91 | 0.233 |
| 19 | 4.66 | 0.168 |
| 20 | 4.11 | 0.198 |

### BRIEF DESCRIPTION OF THE DRAWINGS

FIG. 1 is a photograph showing the claimed hemp plant; and,

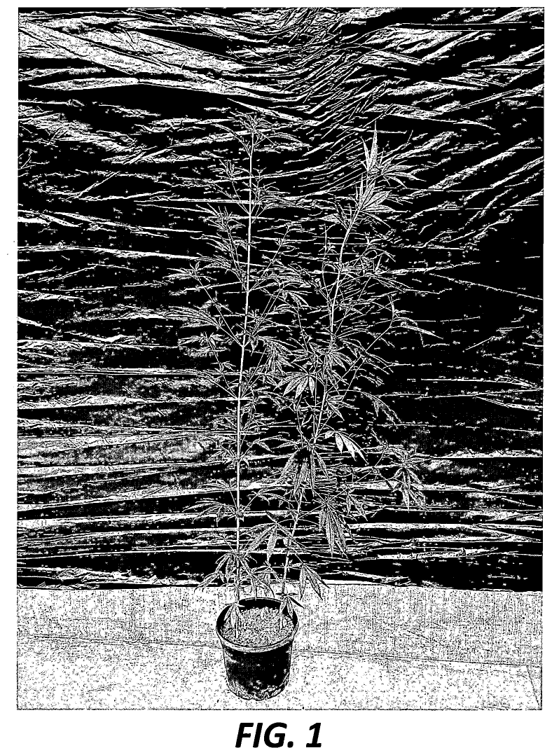

FIG. 2 is a photograph showing the leaves and stalk of the claimed hemp plant.

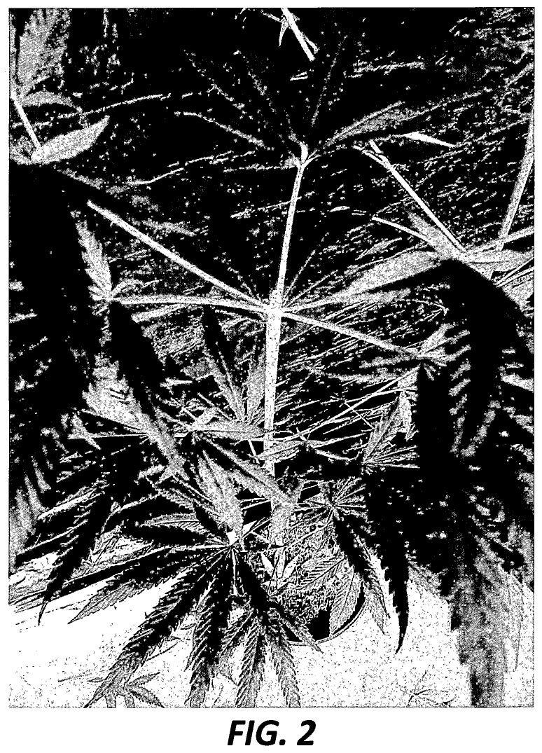

### DETAILED BOTANICAL DESCRIPTION OF THE VARIETY

The patent laws of the United States require that a description for a plant patent be complete and must distinguish the claimed variety. The inventors have sequenced the entirety of the nuclear genome of the ‘CAF#13/2020’ hemp plant, which provides a genotype of the variety at the nucleotide level.

The following methodology was used:
1. Genomic DNA (gDNA) was extracted from leaf material of ‘CAF#13/2020’ hemp plants.
2. A DNA-Seq library was prepared for 150 paired-end sequencing with an Illumina NovaSeq 6000 system.
3. Sequenced reads were checked for quality, and high quality reads were mapped to Cannabis sativa cs10, the representative genome for this species in the National Center for Biotechnology Information (NCBI). The RefSeq assembly accession is GCF_900626175.2 and the assembled genome was downloaded on Dec. 12, 2020.
4. The reads mapping at each nucleotide position were compiled and the corresponding nucleotides were annotated. If a nucleotide position in ‘CAF#13/2020’ had a different nucleotide than that of the reference genome cs10, a single nucleotide variant (SNP) was declared.
5. After applying quality filters to avoid false SNPs, the inventors compiled all SNPs located in genic regions and used to genotype ‘CAF#13/2020’. We found 845,500 high quality SNPs located in genic regions that can be used as a barcode or unique identifier for ‘CAF#13/2020’. The list of these SNPs is compiled in a .bed format file called “CAF#13/2020_genotype.bed”.

This application is accompanied by a compact disc containing the file “CAF#13/2020_genotype.bed” in a compressed format (bed.gz) as a unique identifier for the ‘CAF#13/2020’ hemp plant. Table 2 provides an example of the .bed format:

TABLE 2
Sequence Example

| Line | Column 1 | Column 2 | Column 3 | Column 4 | Column 5 | Column 6 | Column 7 |
|---|---|---|---|---|---|---|---|
| 1 | NC_044371.1 | 40280 | 40280 | A | G | 91 | 0/1 |
| 2 | NC_044371.1 | 40953 | 40953 | G | A | 129 | 1/1 |
| 3 | NC_044371.1 | 245388 | 245388 | A | T, C | 24 | 1/2 |

Referring to Line 1 of Table 2 above, Column 1 (“NC_044371.1”) denotes the chromosome; Column 2 and Column 3 (“40280” and “40280”) identify the start and end chromosome coordinates of the SNP (which, in the case of SNPs, are equal); Column 4 (“A”) identifies the nucleotide in the cs10 reference genome; Column 5 (“G”) identifies the alternative nucleotide found in ‘CAF#13/2020’; Column 6 (“91”) identifies the number of reads mapping at that position; and, Column 7 (“0/1”) the called genotype. The allele values of the genotype are 0 for the cs10 reference allele (“A”) and 1 for the alternative allele (“G”). Thus, the genotype of ‘CAF#13/2020’ at position 40280 of chromosome NC_044371.1 is 0/1 or A/G (i.e., it is heterozygous). By comparison, it can be seen at Line 2 of Table 2 that the genotype of ‘CAF#13/2020’ at position 40953 of chromosome NC_044371.1 is 1/1 or A/A (i.e., it is homozygous for the alternative allele). Finally, there are some cases, such as the example shown in Line 3 of Table 2, where ‘CAF#13/2020’ has two alternative alleles (“T,C”) and the genotype is heterozygous. Homozygous sites in ‘CAF#13/2020’ for the cs10 reference allele are not included in the file. The submitted file includes 605,723 sites with genotype 0/1, 237,831 sites with genotype 1/1 and 1,946 sites with genotype 1/2.

The complete genotype description provided in the accompanying electronic file precisely defines the claimed variety and distinguishes it from other known hemp varieties.

Potency analysis of ‘CAF#13/2020’ was performed by CARO Analytical Services (Richmond, BC, Canada) using test method CR-TM-161 and instrument ID and AIN HPLC CR269-CR274. The analysis confirmed THC concentration of 0.277% by weight, and CBD concentration of 7.380% by weight.

1. A new and distinct hemp plant named ‘CAF#13/2020’ substantially as described herein.
